(19) United States
(12) Patent Application Publication
(10) Pub. No.: US 2021/0069087 A1
(43) Pub. Date: Mar. 11, 2021

(54) PROCESS FOR TREATING HAIR

(71) Applicant: KAO GERMANY GMBH, Darmstadt Hessen (DE)

(72) Inventors: Andreas PICKER, Darmstadt (DE); Andrea AST, Darmstadt (DE); Huma YOUNAS, Darmstadt (DE); Michael MOLENDA, Darmstadt (DE)

(73) Assignee: KAO GERMANY GMBH, Darmstadt Hessen (DE)

(21) Appl. No.: 16/772,287

(22) PCT Filed: Nov. 14, 2018
(86) PCT No.: PCT/EP2018/081164

§ 371 Date: Jun. 12, 2020

(30) Foreign Application Priority Data
Dec. 21, 2017 (EP) 17209516.8

(51) Int. Cl. A61K 8/65; A61K 8/34; A61K 8/362; A61K 8/19; A61K 8/41; A61K 8/20; A61K 8/898; A61K 8/55; A61K 8/98; A61K 8/35; A61Q 5/12

(52) U.S. Cl. CPC A61K 8/65; A61K 8/34; A61K 8/362; A61K 8/19; A61K 8/416; A61Q 5/12; A61K 8/898; A61K 8/55; A61K 8/986; A61K 8/35; A61K 8/415; A61K 8/20

(57) ABSTRACT

The present invention relates to a process for treating hair wherein hair is applied two compositions one after another with an optional rinse off step in order to increase effectiveness of the active compounds comprised in only in the first composition applied onto hair.

The present invention relates to a process for treating hair wherein hair is applied two compositions one after another with an optional rinse off step in order to increase effectiveness of the active compounds.

Hair conditioning has always been practiced for improving one or the other properties of hair and/or enhancing the improved properties obtained with certain treatments. Environmental factors affect hair considerably such as sunlight, exhaust gasses, chemicals released to the environment by the industrial usage, etc. In order to reduce such effects, one way may be depositing effective amounts of protective ingredients onto hair so that although hair is exposed to such environmental damaging factors, due to presence of effective amount of protecting agents its physico-chemistry is kept intact. One example to such may be the effect of sunlight on hair colour. It is known that hair colour is lightened by the effects of sunlight and this may be overcome by depositing effective amount of UV filters onto hair so that light is absorbed by the UV filter and therefore does not affect hair.

It has been the target of the cosmetic product providers for a long time to achieve more and more effective hair treatment methods which not only provide more effect but at the same time it is also easy to use and especially may be combined with other products and/or services.

One of the frequently used hair dressing service is hair colouring, which is usually practiced by application of an highly alkaline composition comprising at the same time an oxidizing agent. Such processes do change hair physico-chemistry, which reflects the appearance and perception of hair quality. On a long run, hair chemistry may be so altered that the hair is not at all perceived as beautiful any more but solely as the part requires more attention. Such kinds of hair are classified as damaged and indeed require more time and products to adjust their perception to a somewhat acceptable level.

Thus there is an urgent need for new hair treatment processes for either preventing excess hair damage and/or for improving and/or reinstating hair beauty which is then perceived as attractive.

EP 1915981 discloses a process for protecting colour of artificially dyed hair with respect to washing and atmospheric effects wherein a hair protecting agent is applied first onto hair which is followed by application of zinc based composition after dyeing hair.

EP1769788 is on a keratin fiber treating process wherein the hair is applied hair colouring composition comprising hair protecting agents such as UV filters.

EP1738739 aims at protecting hair colour by applying composition comprising oil soluble UV filters onto artificially coloured hair and afterwards increasing the hair temperature by means of heating iron to a temperature above 60° C.

After a long research, the inventors of the present invention have found out that application of a composition comprising ingredients which either prevent hair damage or enhance one or the other hair properties, prior to treating hair with an alkaline composition such as hair colour prevents, on the one hand, the hair damage and, on the other hand, improves hair properties such as strength, elasticity, reduced breakage after frequent combing and shine.

The above referred disclosures do not deal with a process of the present invention, as all of them either targeting pre-coloured hair or using the active ingredients within colouring composition. Thus, the process of the present invention is novel over the above summarized prior publications.

Accordingly, the present invention is on a process for treating hair, especially human hair, wherein
hair is applied a composition (Composition A) comprising, in a cosmetically acceptable medium, one or more active ingredients selected from hydrolyzed proteins, amino acids, humectants, radical scavengers, vitamins, ceramides, sterols, essential oils, amino silicones with a molecular weight less than 100,000 and UV filters and/or their respective salts and their mixtures and surfactant at a concentration equal to or less than 3% by weight, calculated to the total of the composition A, and
the composition A is left on the hair for a period of 1 to 60 min,
hair is optionally rinsed off,
hair is applied an aqueous alkaline composition (Composition B) having a pH in the range of 8.5 to 12 and left on the hair for a period 5 to 60 min at a temperature in the range of 20 to 45° C., and
hair is rinsed off and dried.

According to the process of the present invention all of the steps are carried out one after another without any major break.

Further object of the present invention is a kit for hair, especially human hair, comprising the Compositions A and B as outlined above.

The novel and inventive process of the present invention comprising the application of the first composition, Composition A, onto hair comprising one or more active ingredients selected from hydrolyzed proteins, amino acids, humectants, radical scavengers, vitamins, ceramides, sterols, essential oils, amino silicones with a molecular weight less than 100,000 and UV filters and/or their respective salts.

The Composition A may be an aqueous, aqueous alcoholic, alcoholic or an anhydrous composition. The composition A is preferably an aqueous or aqueous-alcoholic composition and comprises preferably 40% or more water, calculated to the total of the Composition A. The Composition A has preferably a pH below and equal to 8.0, more preferably 2 to 7.0, most preferably 3 to 6 and in particular 3.5 to 5.5. The Composition A is preferably a non-foaming composition, in other words, it is not a shampoo composition.

The Composition A comprises one or more active ingredients selected from hydrolyzed proteins, amino acids, humectants, radical scavengers, vitamins, ceramides, sterols, essential oils, amino silicones with a molecular weight less than 100,000 and UV filters and their respective salts at a total concentration in the range of 0.001 to 15%, preferably 0.01 to 12.5%, more preferably 0.05 to 10%, most preferably 0.1 to 7.5% by weight calculated to the total of the composition.

In principal any protein hydrolysate is suitable for the purpose of the present invention. The preferred ones are hydrolyzed wheat protein, hydrolyzed keratin, hydrolyzed milk protein, hydrolyzed silk protein, hydrolyzed soy protein and hydrolyzed avocado protein and their mixtures. The more preferred ones are hydrolyzed keratin, hydrolyzed silk protein and hydrolyzed soy protein and their mixtures. The most preferred are hydrolyzed keratin and hydrolyzed silk protein, and their mixtures. Hydrolyzed keratin is particularly preferred.

The known amino acids and their respective salts are all suitable for the purpose of the present invention. The suitable non-limiting examples are glycine, histidine, citrullin, asparagine, alanine, valine, leucine, isoleucine, proline, tryptophan, phenylalanine, methionine, serine, tyrosine, threonine and glutamine and their respective salts. It should be noted that the Composition A may also comprise mixture of several amino acids.

The Composition A comprises humectants. Suitable ones are all polyols, such as glycerol, propylene glycol, panthenol, phytantriol, lactic acid and its salts, Pyrrolidone carboxylic acids and its salts, sorbitol, and hyaluronic acid and its salts and their mixtures. Prefered are glycerol, panthenol, phytantriol and propylene glycol and their mixtures.

Radical scavengers are comprised in the Composition A. The suitable non-limiting examples are butylhydroxy toluol, tocopheryl nicotinate, flavonoids such as isoquercetin, polyphenold, ascorbic acid and its salts, and cafeic acid and its salts.

Suitable vitamins are Tocopheryl Acetate, Tocopherol (Vitamin E), Biotin (Vitamin B7/Vitamin H), Niacinamide (Vitamin B3 Derviate), Ubiquinone , Pantethine (Vit B5 derivate), Riboflavine (Vit B2), Retinol (Vit A), Carotine, Thiamin (Vit B1), Niacine (Vit B3), Cobalamine (Vit B12), Folic Acid, and Calciferole (Vit D). Ceramides may be comprised in the composition A. Suitable and non-limiting examples are the ones according to the following general structure

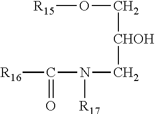

where R₁₅ and R₁₆ are independent from each other an alkyl- or alkenyl group with 10 to 22 carbon atoms, R₁₇ is alkyl or hydroxyl alkyl with 1 to 4 carbon atoms group and n is a number between 1 to 6, preferably 2 or 3. Preferred compound according to the above chemical structure is cetyl-PG-hydroxyethylpalmitamide.

Sterols are comprised as the active ingredient. The non-limiting examples are such as ergosterol, sitosterol, stigmasterol, fucosterol, brassicasterol, fungisterol, campesterol, zymosterol, ascosterol, cerevisterol, episterol, faecosterol and spinasterol. Among those phytosterols, the ones found in “Avocadin” which is the unsaponified fraction of the avocado oil is more preferred

Aminated silicones with a molecular weight 100,000 or lower are one of the preferred active ingredients. Suitable examples are X-52-2265 von Shinetsu with an amine content of 1400-1600 g/mol and a molecular weight of approximately 10,000 g/mol

Further advantageously, aqueous compositions comprise one or more aminated silicones which may be selected from amodimethicones and grafted aminianted siliocnes. Suitable ones are available under various trade names such as DC 969, Belsil from Wacker Chemie AG and know with the CTFA adopted name Amodimethicone, and Elastomer OS from Kao Corporation known with CTFA adopted name Polysilicone-9.)

UV filters are comprised as active ingredients in the Composition A. Suitable ones are both oil and water soluble ones. Non-limiting examples are the following compounds: 4-Aminobenzoic acid and the esters and salts thereof, 2-phenyl benzimidazole-5-sulfonic acid and the alkali and amine salts thereof, 4-dimethyl aminobenzoic acid and the esters and salts thereof, cinnamic acid and the esters and salts thereof, 4-methoxycinnamic acid and the esters and salts thereof, salicylic acid and the esters and salts thereof, 2.4-dihydroxybenzophenone, 2.2′.4.4′-tetrahydroxy-benzophenone, 2-hydroxy-4-methoxybenzophenone and its 5-sulfonic acid or the sodium salt thereof, 2.2′-dihydroxy-4.4′-dimethoxybenzophenone, 2-hydroxy-5-chlorobenzophenone, 2.2′-dihydroxy-4-methoxybenzophenone, 2.2′-dihydroxy-4.4′-dimethoxy-5.5′-disulfobenzo-phenone or the sodium salt thereof, 2-hydroxy-4-octyloxybenzophenone, 2-hydroxy-4-methoxy-4′-methylbenzophenone, 3-benzyl-idenecampher, 3-(4′-sulfo)-benzyl-idenebornane-2-one and the salts thereof and/or 3-(4′-methyl benzylidene)-DL-campher.

The composition A comprises one or more organic solvent, preferably selected from aliphatic alcohols such as ethanol, isopropanol, propanol, n-butanol, isobutanol, t-butanol and 1-pentanol, 2-phenoxyethanol, benzyl alcohol, 2-phenylethanol and 2-benzyloxyethanol. Total concentration of one or more organic solvent is in the range of 1 to 55%, preferably 2 to 50% and more preferably 5 to 40% and most preferably 10 to 40% by weight calculated to the total the composition A.

The pH of the Composition A may be adjusted with the known organic and/or inorganic acids and bases.

The composition A is left on the hair for a period of 1 to 60 min, preferably 2 to 45 min, more preferably 5 to 30 min and most preferably 5 to 15 min on the hair preferably at a temperature in the range of 20 to 45° C. In the preferred embodiment, the composition A is not rinsed off from hair prior to application of the Composition B.

The composition A may be in any product form such as a solution which may be applied by using a pump spray, a thickened gel and an emulsion. Each of the composition may also be made available in a pressurized container in the form of an aerosol composition dispensed as spray or foam.

The composition B is an aqueous alkaline composition having a pH in the range of 8.5 to 12. Preferably the pH of the composition is in the range of 9 to 11, more preferably 9 to 10.5 and most preferably 9.5 to 10.5.

The composition comprises one or more alkalizing agent. Suitable alkalizing agents are the alkali hydroxides such as sodium hydroxide, potassium hydroxide, ammonia and its salts such as ammonium carbonate, ammonium hydrogen carbonate, ammonium carbamate, ammonium chloride, ammonium sulfate, ammonium phosphates such as ammonium dihydrogen phosphate, diammonium hydrogen phosphate, diammonium sodium phosphate, ammonium sodium hydrogen phosphate or ammonium disodium phosphate, ammonium nitrate, ammonium bromide, ammonium iodide, ammonium thiosulfate, ammonium molybdate, ammonium vanadate, ammonium sulfamate, ammonium citrate, ammonium salicylate, ammonium valerate, ammonium tartrate, ammonium benzoate, ammonium acetate, ammonium formiate and ammonium lactate, guanidine and its salts such as guanidine hydrochloride, guanidine carbonate, guanidine bicarbonate, and an alkyl or alkanol amine according to the general structure

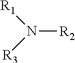

wherein R₁, R₂, and R₃ are same or different H, from C₁ to C₄, C₃ to C₄ unsaturated alkyl, C₃ to C₄ branched alkyl, C₁ to C₄ hydroxyl alkyl, C₃ to C₄ unsaturated hydroxyl alkyl, C₃ to C₄ branched hydroxyl alkyl, with the condition that at least one of R₁, R₂, or R₃ is different from H, such as monoethanolamine, diethanolamine, triethanolamine, monoethanol methylamine, monoethanoldimethylamine, diethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine and amino methyl propanol and their mixtures.

Preferred are sodium and potassium hydroxides, ammonia and its salts, monoethanolamine, diethanolamine, triethanolamine, amino methyl propanol, guanidine salts and their mixtures. The most preferred are ammonia, ammonium chloride, guanidine carbonate, monoethanolamine and amino methyl propanol and their mixtures.

The total concentration of one or more alkalizing agent is in the range of 0.1 to 15%, preferably 0.25 to 12.5%, more preferably 0.5 to 10%, most preferably 1 to 7.5% by weight calculated to the total of the composition B.

The composition B may further comprise an oxidizing agent, preferably hydrogen peroxide, preferably at a concentration in the range of 0.1 to 15%, preferably 0.2 to 12.5%, more preferably 0.25 to 10% and most preferably 0.5 to 8% by weight calculated to the total of the aqueous composition B.

In case the purpose of the process is colouring hair, the Composition B comprises one or more hair dyes. Suitable ones are direct dyes and/or oxidative dyes.

One or more hair direct dyes are selected from anionic, cationic and neutral ones.

Suitable anionic direct dyes are Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2, D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as sodium, potassium. Among those, the most preferred anionic dyestuffs are Acid Red 52, DC Violet 2, DC Red 33, DC Orange 4, DC Red 27, DC Yellow 10, HC Blue 18, HC Red 18, and HC Yellow 16.

Suitable cationic dyes are in principle those available on the market for cosmetic hair colouring applications. For this purpose, special reference is made to the PCT application WO 95/15144 of Ciba-Geigy AG. Some examples to those are Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Red 2, Basic Red 12 Basic Red 22, Basic Red 76, Basic Violet 1, Basic Violet 2, Basic Violet 3, Basic Violet 10, Basic Violet 14, Basic Yellow 57, Basic Red 51, Basic Yellow 87, HC Blue 17 and Basic Orange 31. The most preferred ones are Basic Red 51, Basic Yellow 87 Basic Orange 31, HC Blue 17 and Basic Blue 124.

Suitable neutral dyes including nitro dyes are HC Blue No.2, HC Blue No.4, HC Blue No.5, HC Blue No.6, HC Blue No.7, HC Blue No.8, HC Blue No.9, HC Blue No.10, HC Blue No.11, HC Blue No.12, HC Blue No.13, HC Brown No.1, HC Brown No.2, HC Green No.1, HC Orange No.1, HC Orange No.2, HC Orange No.3, HC Orange No.5, HC Red BN, HC Red No.1, HC Red No.3, HC Red No.7, HC Red No.8, HC Red No.9, HC Red No.10, HC Red No.11, HC Red No.13, HC Red No.54, HC Red No.14, HC Violet BS, HC Violet No.1, HC Violet No.2, HC Yellow No.2, HC Yellow No.4, HC Yellow No.5, HC Yellow No.6, HC Yellow No.7, HC Yellow No.8, HC Yellow No.9, HC Yellow No.10, HC Yellow No.11, HC Yellow No.12, HC Yellow No.13, HC Yellow No.14, HC Yellow No.15, 2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid.

The composition comprises one or more hair direct dyes at a total concentration of 0.001% to 10%, preferably 0.005% to 9%, and more preferably 0.01% to 7.5% by weight, calculated to the total of the composition B. The composition can also comprise a mixture of several direct dyes, i.e., anionic, cationic and/or nonionic ones. In such a case the dyes may be mixed at any ratio with each other.

The composition B comprises one or more oxidative dye precursors and optionally one or more coupling substances.

Suitable oxidative dye precursors classes are p-phenylendiamines, p-aminophenols, and heterocyclic compounds such as diaminopyrazols and substituted pyrimidines, and suitable coupling substances are resorcinols, m-aminophenols, m-phenylendiamines, pyridines and substituted derivatives, and naphthols.

Non-limiting examples of the oxidative dye precursor compounds are p-phenylenediamine, p-aminophenol, 2,5-diamino-toluene, 2-n-propyl or 2-ethyl-p-phenylenediamine, 2,6-di-methyl-p-phenylene-diamine, 2-(2,5-diaminophenyl) ethanol, 1-amino-4-bis-(2′-hydroxy-ethyl)amino-benzene, 2-(2-hydroxyethyl amino)-5-aminotoluene, 4,4′-diaminodiphenylamine, 4-aminodiphenylamine, 2-amino-5-N,N-diethyl aminotoluene, 4-amino-N-ethyl-N-isopropyl aniline, 2-chloro-p-phenylenediamine, 1-β-hydroxyethyl-2,5-diamino-4-chlorobenzene, 1-β-hydroxyethyl-2,5-diamino-4-methyl benzene, 2-methoxy-p-phenylenediamine, N,N-diethyl-p-phenylenediamine, 1-amino-4-β-methoxyethyl aminobenzene, 1-dimethyl-amino-4-aminobenzene, 1-hydroxy-2,5-diamino-4-methyl benzene, 1-hydroxymethyl-2,5-diaminobenzene, 1,3-dimethyl-2,5-diaminobenzene, 1,4-diamino isopropyl benzene and/or 1-amino-4-β-hydroxypropyl aminobenzene, 1-hydroxyethyl-4,5-diaminopyrazole, 3,4-diamino-5-hydroxypyrazole, 3,5-diaminopyrazole, 3,5-diamino pyrazol-1-carboxamide, 3-amino-5-hydroxypyrazole, 1-phenyl-2-methylpyrazole, 1-phenyl-3-methylpyrazole-5-one, 3,5-dimethylpyrazole, 3,5-dimethylpyrazole-1-methanol, 3,5-diamino-1,2,4-triazole, 4-aminophenol and the derivatives thereof such as 4-amino-3-methylphenol, 2-chloro-4-aminophenol, 2,6-dichloro-4-aminophenol, 2,4-diamino-phenol, 2,6-dibromo-4-aminophenol, tetraamino pyrimidines, triaminohydroxy pyrimidines, diaminomono- and -dihydroxy pyrimidines, aminotriazines, 5-amino salicylic acid and/or 1,2,4-triamino benzene, 2,5-diaminopyridine, 2,3-diaminopyridine, 2,6-diaminopyridine, 3-amino-2-methyl amino-6-methoxypyridine, 2-dimethyl-5-aminopyridine, 2-dimethyl aminoethyl-3-hydroxypyridine, 2-amino-4,6-dimethyl pyridine, 2-amino-3-hydroxypyridine, 3-amino-2(β-hydroxyethyl amino)-6-methoxy pyridine, 2,6-dimethyl amino-5-aminopyridine, 2-dihydroxyethyl) amino-5-aminopyridine, 2-hydroxyethyl amino-5-aminopyridine, 4-hydroxy-2,5,6-triaminopyrimidine and/or the water-soluble salts thereof, and mixture thereof.

The total concentration of the dye precursors (developing substances) customarily ranges between 0.001 to 5%, preferably 0.01 to 4% and more preferably 0.05 to 3%, and most preferably 0.1 to 2% by weight, calculated to the total of the composition B.

The aqueous composition comprising the oxidative dye precursor may further comprise coupling substances. The suitable non-limiting examples of the coupling substance if present in the composition A are 5-amino-2-methylphenol, 2-methyl-5-hydroxyethylaminophenol, 2,4,-diaminophenoxyethanol, 2-amino-4-hydroxyethylaminoanisol, 2-methyl-5-amino-6-chlorphenol, 1,3-bis(2,4-diaminophenoxy)propane, 2-bis(2-hydroxyethyl)aminotoluene, 2-amino-5-methylphenol, resorcinol, 2-methyl-resorcinol, 4-chlororesorcinol, 2-amino-4-chlorophenol, 5-amino-4-methoxy-2-methylphenol, 2-aminophenol, 3-amino-phenol, 1-methyl-2-hydroxy-4-aminobenzene, 3-N,N-dimethyl aminophenol, 2,6-dihydroxy-3,5-dimethoxypyridine, 5-amino-3-methylphenol, 6-amino-3-methylphenol, 1,3-diamino-benzene, 1-amino-3-(2′-hydroxyethylamino)benzene, 1-amino-3-[bis(2′-hydroxy-ethyl) amino]benzene, α-naphthol, 4,6-dichlororesorcinol, 1,3-diamino-toluene, 4-hydroxy-1,2-methylenedioxy benzene, 1,5-dihydroxy naphthalene, 1,6-dihydroxy naphthalene, 1,7-dihydroxy naphthalene, 2,7-dihydroxy naphthalene, 1-hydroxy-2-methyl naphthalene, 4-hydroxy-1,2-methyldioxy benzene, 2,4-diamino-3-chlorophenol, 5-amino-2-methoxyphenol and/or 1-methoxy-2-amino-4-(2′-hydroxyethyl amino)benzene or the water-soluble salts thereof and mixture thereof.

The coupling substances are present in approximately the same molecular proportions as the developing substances, i.e. at a total concentration in the range of 0.001 to 5%, preferably 0.01 to 4% and more preferably 0.05 to 3%, and most preferably 0.1 to 2% by weight, calculated to the total of the composition B.

In the following, all reported concentrations must be understood as relative to each of the compositions because, firstly, the compositions are not mixed with each other and secondly, the same ingredient disclosed must not be comprised in all of the composition, although this is a possibility.

The compositions A and/or B advantageously comprise a thickening agent, preferably a thickening polymer. Suitable and preferred ones are thickening polymers such as polysaccharides such as alginate, pectinate, xanthan, hydroxypropyl xanthan or dehydroxanthan, non-ionic polysaccharides such as cellulose ethers (e.g., methylcellulose, hydroxyethylcellulose (HEC), methyl hydroxyethylcellulose (MHEC), ethyl hydroxyethylcellulose (EHEC), methyl ethyl hydroxyethylcellulose (MEHEC)), starch or dextrins. Synthetic acrylate type of thickeners may as well be comprised such as acrylate copolymers and alkyl acrylates homo or copolymers also known as associative thickeners.

The concentration of the thickening polymer is very much dependent on the type of the thickening polymer and the targeted consistency (viscosity) of the compositions. Typically, the thickening polymers are comprised in the compositions at a concentration in the range of 0.1 to 3%, preferably 0.25 to 2% by weight calculated to the total of each of the compositions A or B.

The compositions A and/or B can comprise one or more fatty alcohols. Suitable fatty alcohols are the ones with the chain length of 14 to 22 C atoms which may be saturated or unsaturated, linear or branched which may as well be substituted. Non-limiting examples are myristyl alcohol, cetyl alcohol, stearyl alcohol, behenyl alcohol, octyl dodecanol, cetostearyl alcohol, and their mixtures.

The total concentration of fatty alcohol is in the range from 0.5 to 15%, preferably 1 to 10% by weight, calculated to total of each of the compositions A or B. The compositions A and/or B advantageously comprise one or more surfactants wherein the composition A comprises surfactants at a concentration 3% by weight or less calculated to the total of the composition A. Suitable ones are selected from anionic, non-ionic, amphoteric and cationic ones.

Anionic surfactants suitable are in principle known from the cleansing compositions. These are anionic surfactants of the sulfate, sulfonate, carboxylate and alkyl phosphate type, for example, the known C₁₀-C₁₈-alkyl sulfates, and in particular the respective ether sulfates, for example, C₁₂-C₁₄-alkyl ether sulfate, lauryl ether sulfate, especially with 1 to 4 ethylene oxide groups in the molecule, monoglyceride (ether) sulfates, fatty acid amide sulfates obtained by ethoxylation and subsequent sulfatation of fatty acid alkanolamides, and the alkali salts thereof, as well as the salts of long-chain mono- and dialkyl phosphates. Preferred anionic surfactants are alkyl sulphate surfactants especially lauryl sulphate and its salts.

Further suitable surfactants are nonionic surfactants. Non-limiting examples are long-chain fatty acid mono- and dialkanolamides, such as coco fatty acid mono- or diethanolamide and myristic fatty acid mono or diethanolamide, stearic acid mono or diethanolamide, alkyl polyglucosides with an alkyl group of 8 to 18 carbon atoms, and with 1 to 5 glucoside units, sorbitan esters, such as polyethylene glycol sorbitan stearic, palmitic, myristic and lauric acid esters, fatty acid polyglycol esters or poly-condensates of ethyleneoxide and propyleneoxide, as they are on the market, for example, under the trade name “Pluronics^{R}”, as well as fatty alcohol ethoxylates, C₁₀-C₂₂-fatty alcohol ethoxylates, known by the generic terms “Laureth”, “Myristeth”, “Oleth”, “Ceteth”, “Deceth”, “Steareth” and “Ceteareth” according to the CTFA nomenclature, including addition of the number of ethylene oxide molecules, e.g., “Laureth-16”: The average degree of ethoxylation thereby ranges between about 2.5 and about 100, preferably about 10 and about 30.

Suitable amphoteric surfactants are in particular the various known betaines such as alkyl betaines, fatty acid amidoalkyl betaines and sulfobetaines, for example, lauryl hydroxysulfobetaine; long-chain alkyl amino acids, such as cocoaminoacetate, cocoaminopropionate and sodium cocoamphopropionate and -acetate have also been proven suitable.

Suitable cationic surfactants are according to the general structure

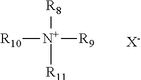

wherein R₈ is a saturated or unsaturated, branched or linear alkyl chain with 8-22 C atoms or

R₁₂ CO NH (CH₂)ₙ

where R₁₂ is saturated or unsaturated, branched or linear alkyl chain with 7-21 C atoms and n has typical value of 1-4 or

R₁₃ CO O (CH₂)ₙ

where R₁₃ is saturated or unsaturated, branched or linear alkyl chain with 7-21 C atoms and n has typical value of 1-4, and

R₉ is H or unsaturated or saturated, branched or linear alkyl chain with 1-22 C atoms or

R₁₂ CO NH (CH₂)ₙ

or

R₁₃ CO O (CH₂)ₙ

where R₁₂, R₁₃ and n are same as above.

R₁₀ and R₁₁ are H or lower alkyl chain with 1 to 4 Carbon atoms, and X is typically chloride, bromide, methosulfate.

Typical examples of those ingredients are cetyl trimethyl ammonium chloride, stearyl trimonium chloride, dipalmitoyl dimonium chloride, distearyl dimethyl ammonium chloride, stearamidopropyl trimonuim chloride, dioleoylethyl dimethyl ammonium methosulfate, dioleoylethyl hydroxyethylmonium methosulfate.

The Composition A comprises 3% by weight or less surfactant, calculated to the total of the composition A. In Composition A, the total concentration of one or more surfactants is in the range of 0.1 to 2.5%, preferably 0.2 to 2% and more preferably 0.25-1.5% by weight, calculated to the total of the Compositions A.

In Composition B, the total concentration of one or more surfactants is in the range of 0.1 to 12.5%, preferably 0.2 to 10% and more preferably 0.5-7.5% by weight, calculated to the total of the Compositions B.

Further advantageously, compositions A and/or B comprise one or more silicone compound, preferably silicone oil. Suitable and preferred ones are known with their CTFA adopted name as dimethicone and commercially available from Dow Corning under the trade name DC 200 with various viscosities.

Further advantageously, the compositions comprise one or more cationic polymers as conditioning and/or thickening agents. Those are cationic cellulose type polymers know as Polymer JR type from Amerchol such as Polyquaternium 10 or cationic guar gum known with trade name Jaguar from Rhone-Poulenc and chemically for example Guar hydroxypropyl trimonium chloride. Furthermore, chitosan and chitin can also be included in the compositions as cationic natural polymers.

Furthermore, it has been found suitable those cationic polymers known with their CTFA category name Polyquaternium. Typical examples of those Polyquaternium 4, Polyquaternium 6, Polyquaternium 7, Polyquaternium 10, Polyquaternium 11, Polyquaternium 16, Polyquaternium 22, Polyquaternium 24, Polyquaternium 28, Polyquaternium 30, Polyquaternium 37, Polyquaternium 36, Polyquaternium 46, Polyquaternium 67, and Polyquaternium 72.

The total concentration of cationic polymers may be in the range of 0.1-2.5%, preferably 0.25-2% by weight and more preferably 0.5-1.5% by weight, calculated to total of each of the compositions A or B.

Any of the compositions described in detail above may comprise ingredients customarily found in such compositions such as preservative, fragrance, chelating agents, etc.

Following examples are to illustrate the invention but not to limit it.

### EXAMPLE 1

The four Fischbach Miller hair tresses were prepared as follows:

The tress 1 was kept as untreated reference hair tress.

The tress 2 was Colored with oxidative hair dyeing composition available under the brand Goldwell Topchic shade 6N. Therefore, the Topchic 6N was mixed 6% Topchic Oxidizing Lotion at a weight ratio of 1:1 and applied onto hair at a weight ratio of hair to composition of 1 to 4. After 30 minutes of processing at 40° C., tress was rinsed and shampooed with Goldwell Kerasilk Color Gentle Sulfate Free Shampoo.

The tress 3 was treated with pre-treatment for a period 5 min not comprising keratin hydrolysate as outlined below. Afterwards, without rinsing off the pretreatment, the tress was colored with oxidative hair dyeing composition available under the brand Goldwell Topchic shade 6N. Therefore, the Topchic 6N was mixed 6% Topchic Oxidizing Lotion at a weight ratio of 1:1 and applied onto hair at a weight ratio of hair to composition of 1 to 4. After 30 minutes of processing at 40° C., tress was rinsed and shampooed with Goldwell Kerasilk Color Gentle Sulfate Free Shampoo.

The tress 3 was treated with pre-treatment for a period 5 min comprising keratin hydrolysate as outlined below. Afterwards, without rinsing off the pretreatment, the tress was colored with oxidative hair dyeing composition available under the brand Goldwell Topchic shade 6N. Therefore, the Topchic 6N was mixed 6% Topchic Oxidizing Lotion at a weight ratio of 1:1 and applied onto hair at a weight ratio of hair to composition of 1 to 4. After 30 minutes of processing at 40° C., tress was rinsed and shampooed with Goldwell Kerasilk Color Gentle Sulfate Free Shampoo.

Pre-Treatment Composition

| | % by weight |
|---|---|
| Hydrolysed Keratin* | 5.0 |
| Ethanol | 35.0 |
| Citric acid/Sodium hydroxide | q.s. to pH 4.8 |
| Water | to 100 |

*added as a raw material CROTEIN CASHMERE PE-LQ from Croda which comprises 17.5% Hydrolyzed Keratin and sodium chloride, potassium sorbate and phenoxyethanol at lower concentrations.

For all tresses, cysteic acid content of the hair was analyzed with an amino acid analyzer according to the method well known in the art. The following results were obtained.

| | Cysteic acid (μmol/g hair) |
|---|---|
| Tress 1 | 42.1 |
| Tress 2 | 107.9 |
| Tress 3 | 99.5 |
| Tress 4 | 91.2 |

From the above results, it is beyond any doubt that oxidative hair colouring increases the cysteic acid content of the hair. The pre-treatment with a composition without keratin hydrolysates showed slight decrease in the cysteic acid content of the hair. The clear decrease in the cysteic acid content of hair was observed with the composition comprising keratin hydrolysate.

### EXAMPLE 2

Human hair tress (Tress 5) weighting approximately 3 g having a Level 9 natural colour was coloured with oxidative hair dyeing composition available under the brand Goldwell Topchic shade 4B. Therefore, the Topchic 4B was mixed 6% Topchic Oxidizing Lotion at a weight ratio of 1:1 and applied onto hair at a weight ratio of hair to composition of 1 to 4. After 30 minutes of processing at 40° C., tress was rinsed and shampooed with Goldwell Kerasilk Color Gentle Sulfate Free Shampoo.

Human hair tress (Tress 6) weighting approximately 3 g having a Level 9 natural colour was was treated with pre-treatment for a period of 10 min comprising keratin hydrolysate as outlined above and afterwards without rinsing off the pre-treatment composition coloured with oxidative hair dyeing composition available under the brand Goldwell Topchic shade 4B. Therefore, the Topchic 4B was mixed 6% Topchic Oxidizing Lotion at a weight ratio of 1:1 and applied onto hair at a weight ratio of hair to composition of 1 to 4. After 30 minutes of processing at 40° C., tress was rinsed and shampooed with Goldwell Kerasilk Color Gentle Sulfate Free Shampoo.

Human hair tress (Tress 7) weighting approximately 3 g having a Level 9 natural colour was coloured with oxidative hair dyeing composition available under the brand Goldwell Topchic shade 4B which is added keratin hydrolysate at the same comcnetration as for Tress 6. Therefore, the Topchic 4B was mixed 6% Topchic Oxidizing Lotion at a weight ratio of 1:1 and mixed with keratin hydrolysate and applied onto hair at a weight ratio of hair to composition of 1 to 4. After 30 minutes of processing at 40° C., tress was rinsed and shampooed with Go!dwell Kerasilk Color Gentle Sulfate Free Shampoo.

The resistance to mechanical external damage stress was determined with an automatic combing machine available from Hercules Sagemann. Therefore, the tresses were combed at both sides for 15000 times (in total 30000 times per tress) at a speed of 1 combing per second and the broken hair fibers were collected and weighted. The following results were obtained.

| | Broken hair amount in mg |
|---|---|
| Tress 5 | 53 |
| Tress 6 | 12 |
| Tress 7 | 31 |

From the above results it is beyond any doubt that significantly less hair was collected as broken from the tress (Tress 6) pre-treated with the composition comprising the keratin hydrolysate compared to the ones either not treated with protein hydrolysate at all (tress 5) or the keratin hydrolysate comprised in the colouring composition (Tress 7).

The following examples are within the scope of the present invention.

### EXAMPLE 3

### Composition A

| | |
|---|---|
| Alcohol | 32% |
| Cetrimonium Chloride | 0.3% |
| Sodium Chloride | 1.0% |
| Amodimethicone | 0.3% |
| Polyquaternium-10 | 0.25% |
| Hydrolized Keratin | 5.0% |
| Sodium Benzoate | 0.5% |
| Phosphoric acid | q.s. pH 4.0 |
| Water | add to 100% |

### EXAMPLE 4

### Composition A

| | |
|---|---|
| Hydrolized Milk Protein | 5.0% |
| Sodium Benzoate | 0.5% |
| Citric acid | q.s. pH 4.2 |
| Water | add to 100% |

### EXAMPLE 5

### Composition A

| | |
|---|---|
| Benzophenone-3 | 5.0 |
| n-Propanol | 15% |
| Phosphoric acid/Sodium hydroxide | q.s. to pH 5.0 |
| Water | add to 100% |

### EXAMPLE 6

### Composition A

| | |
|---|---|
| Benzophenone-4 | 5.0% |
| Sodium Benzoate | 0.5% |
| Phosphoric acid/NaOH | q.s. to pH 3.8 |
| Water | add to 100% |

### EXAMPLE 7

### Composition B

| | |
|---|---|
| Cetearyl Alcohol | 5.0% |
| Ceteareth-20 | 1.5% |
| Phenoxyethanol | 0.27% |
| Ethylhexylglycerin | 0.03% |
| Sodium Hydroxide | adjust pH 10.2 |
| Water | add to 100% |

### EXAMPLE 8

### Composition B

| | |
|---|---|
| Cetearyl Alcohol | 14.0% |
| Sodium Cetearyl Sulfate | 2.0% |
| Cocamide MEA | 3.0% |
| Toluene-2,5-Diamine Sulfate | 1% |
| m-Aminophenol | 0.3% |
| 2-Amino-3-Hydroxypyridine | 0.3% |
| 2,4-Diaminophenoxyethanol HCl | 0.3% |
| Ammonium Hydroxide | 2.2 |
| Water | add to 100% |

The above composition had a pH 10.2. The above composition was mixed with an aqueous composition comprising 6% by weight hydrogen peroxide at a weight ratio of 1:1 and applied onto hair.

### EXAMPLE 9

### Composition B

| | |
|---|---|
| Cetearyl Alcohol | 14.0% |
| Sodium Cetearyl Sulfate | 2.0% |
| Cocamide MEA | 3.0% |
| Toluene-2,5-Diamine Sulfate | 1% |
| m-Aminophenol | 0.3% |
| 2-Amino-3-Hydroxypyridine | 0.3% |
| 2,4-Diaminophenoxyethanol HCl | 0.3% |
| Monoethanolamine | 6.2 |
| Water | add to 100% |

The above composition had a pH 10.5. The above composition was mixed with an aqueous composition comprising 6% by weight hydrogen peroxide at a weight ratio of 1:1 and applied onto hair.

### EXAMPLE 10

### Composition B

| | |
|---|---|
| Cetearyl Alcohol | 14.0% |
| Sodium Cetearyl Sulfate | 2.0% |
| Cocamide MEA | 3.0% |
| Toluene-2,5-Diamine Sulfate | 1% |
| m-Aminophenol | 0.3% |
| 2-Amino-3-Hydroxypyridine | 0.3% |
| Basic red 51 | 0.1 |
| Basic orange 31 | 0.1 |
| Basic yellow 87 | 0.1 |
| HC Red 3 | 0.1 |
| Basic red 76 | 0.1 |
| 2,4-Diaminophenoxyethanol HCl | 0.3% |
| Monoethanolamine | 6.2 |
| Water | add to 100% |

The above composition had a pH 10.1. The above composition was mixed with an aqueous composition comprising 6% by weight hydrogen peroxide at a weight ratio of 1:1 and applied onto hair.

1. A process for treating hair, comprising the steps of, in order
applying to hair a composition A comprising, in a cosmetically acceptable medium, one or more active ingredients selected from hydrolyzed proteins, amino acids, humectants, radical scavengers, vitamins, ceramides, sterols, essential oils, amino silicones with a molecular weight less than 100,000 and UV filters and/or their respective salts and their mixtures, and surfactant; wherein if surfactant is present it is at a concentration equal to or less than 3% by weight, calculated to the total of the composition A, and
leaving the composition A on the hair for a period of 1 to 60 min,
either rinsing composition A from the hair or leaving composition A on the hair,
applying to the hair an aqueous alkaline composition B having a pH in the range of 8.5 to 12 and
leaving composition B on the hair for a period 5 to 60 min at a temperature in the range of 20 to 45° C., and
rinsing composition B from the hair and drying the hair.

2. The process according to claim 1 wherein the Composition A is an aqueous, aqueous-alcoholic, alcoholic or an anhydrous composition.

3. The process according to claim 1 wherein the Composition A has a pH below or equal to 8.0.

4. The process according to claim 1 wherein the Composition A comprises one or more active ingredients at a total concentration in the range of 0.001 to 15% by weight calculated to the total of the composition A.

5. The process according to claim 1 comprising the step of leaving Composition A on the hair prior to applying Composition B.

6. The process according to claim 1 wherein the one or more active ingredients in the Composition A is hydrolyzed protein which is selected from hydrolyzed wheat protein, hydrolyzed keratin, hydrolyzed milk protein, hydrolyzed silk protein, hydrolyzed soy protein and hydrolyzed avocado protein and their mixtures or is a UV filter or is a ceramide according to the following general structure 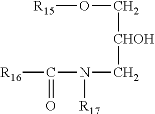
where R₁₅ and R₁₆ are independent from each other an alkyl- or alkenyl group with 10 to 22 carbon atoms, R₁₇ is alkyl or hydroxyl alkyl with 1 to 4 carbon atoms group and n is a number between 1 to 6.

7. The process according to claim 1 wherein the Composition A comprises one or more organic solvent, selected from ethanol, isopropanol, propanol, n-butanol, isobutanol, t-butanol and 1-pentanol, 2-phenoxyethanol, benzyl alcohol, 2-phenylethanol and 2-benzyloxyethanol, at a total concentration in the range of 1 to 55% by weight calculated to the total the composition A.

8. The process according to claim 1 wherein the Composition B comprises one or more alkalizing agents selected from alkali hydroxides such as sodium hydroxide, potassium hydroxide, ammonia and its salts such as ammonium carbonate, ammonium hydrogen carbonate, ammonium carbamate, ammonium chloride, ammonium sulfate, ammonium phosphates such as ammonium dihydrogen phopshate, diammonium hydrogen phosphate, diammonium sodium phosphate, ammonium sodium hydrogen phosphate or ammonium disodium phosphate, ammonium nitrate, ammonium bromide, ammonium iodide, ammonium thiosulfate, ammonium molybdate, ammonium vanadate, ammonium sulfamate, ammonium citrate, ammonium salicylate, ammonium valerate, ammonium tartarate, ammonium benzoate, ammonium acetate, ammonium formiate and ammonium lactate, guanidine and its salts such as guanidine hydrochloride, guanidine carbonate, guanidine bicarbonate, and an alkyl or alkanol amine according to the general structure 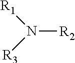
wherein R₁, R₂, and R₃ are same or different H, from C₁ to C₄, C₃ to C₄ unsaturated alkyl, C₃ to C₄ branched alkyl, C₁ to C₄ hydroxyl alkyl, C₃ to C₄ unsaturated hydroxyl alkyl, C₃ to C₄ branched hydroxyl alkyl, with the condition that at least one of R₁, R₂, or R₃ is different from H, such as monoethanolamine, diethanolamine, triethanolamine, monoethanol methylamine, monoethanoldimethylamine, di-ethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine and amino methyl propanol and their mixtures.

9. The process according to claim 1 wherein the total concentration of one or more alkalizing agent is in the range of 0.1 to 15%, by weight calculated to the total of the composition B.

10. The process according to claim 1 wherein the composition B comprises an oxidizing agent at a concentration in the range of 0.1 to 15%, by weight calculated to the total of the aqueous composition B

11. The process according to claim 1 wherein the Composition B comprises one or more hair dyes selected from direct dyes, selected from anionic, cationic and neutral ones and/or oxidative dyes.

12. The process according to claim 1 wherein one or both of the compositions A and B comprises one or more of the thickening agent, a thickening polymer and/or one or more fatty alcohols.

13. The process according to claim 1 wherein one or both of the compositions A and B comprises one or more surfactants selected from anionic, non-ionic, amphoteric and cationic ones, wherein the composition A comprises one or more surfactants at a total concentration in the range of 0.1 to 2.5% by weight, calculated to the total of the Compositions A, and wherein the Composition B comprises one or more surfactants at a total concentration in the range of 0.1 to 12.5% by weight, calculated to the total of the Compositions B.

14. The process according to claim 1 wherein one or both of the compositions A and B comprises cationic surfactants according to the general structure 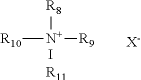
wherein R₈ is a saturated or unsaturated, branched or linear alkyl chain with 8-22 C atoms or
R₁₂ CO NH (CH₂)ₙ
where R₁₂ is saturated or unsaturated, branched or linear alkyl chain with 7-21 C atoms and n has typical value of 1-4 or
R₁₃ CO O (CH₂)ₙ
where R₁₃ is saturated or unsaturated, branched or linear alkyl chain with 7-21 C atoms and n has typical value of 1-4, and
R₉ is H or unsaturated or saturated, branched or linear alkyl chain with 1-22 C atoms or
R₁₂ CO NH (CH₂)ₙ
or
R₁₃ CO O (CH₂)ₙ
where R₁₂, R₁₃ and n are same as above.
R₁₀ and R₁₁ are H or lower alkyl chain with 1 to 4 Carbon atoms, and X is typically chloride, bromide, methosulfate.

15. Kit for hair, especially human hair, comprising
a composition A comprising, in a cosmetically acceptable medium, one or more active ingredients selected from hydrolyzed proteins, amino acids, humectants, radical scavengers, vitamins, ceramides, sterols, essential oils, amino silicones with a molecular weight less than 100,000 and UV filters and/or their respective salts and their mixtures, and surfactant, wherein when surfactant is present it is at a concentration equal to or less than 3% by weight, calculated to the total of the composition A, and
a composition B comprising an aqueous alkaline composition.
